(19) United States
(12) Patent Application Publication
(10) Pub. No.: US 2010/0310636 A1
(43) Pub. Date: Dec. 9, 2010

(54) STEARICALLY STABILIZED UNILAMILAR VESICLES, PROCESS FOR PREPARATION THEREOF AND USE THEREOF

(76) Inventors: Gita Sharma, Gujarat (IN); Chatan Majumdar, Gujarat (IN); Surendra Chikara, Gujarat (IN); Abhijit Mehta, Gujarat (IN)

Correspondence Address: Komal Shah;InnovarIP Consulting Group, 3rd Floor Bhukhanvala Chambers,, B-28 Veera Industrial Estate Andheri West, Mumbai, 400053, IN

(21) Appl. No.: 12/599,290

(22) PCT Filed: May 5, 2008
(86) PCT No.: PCT/IN08/00282

§ 371 Date: Dec. 7, 2009

(30) Foreign Application Priority Data
May 9, 2007 (IN) 885/MUM/2007

(51) Int. Cl. A61K 9/133; A61K 38/18; A61P 7/06

(52) U.S. Cl. 424/450; 514/7.7; 977/773

(57) ABSTRACT

The present invention relates to modified formulations of recombinant human glycoprotein having an in-vivo biological activity to increase the production of reticulocytes and red blood cells. A stearically stabilized unilamillar vesicles (SSUV) containing the said biopharmaceutical composition and process for preparation thereof is provided. The pharmaceutical composition comprises a various lipids, covalently modified lipids with polyethylene glycol and or neutral detergent to form long circulating and tightly packed lipid vesicles, which reduced the reticulo-endothelial clearance of SSUV and cause the sustained released effect of encapsulated biopharmaceutical and recombinant human glycoprotein or an erythropoietin moiety. The scope of present invention also describes the process of encapsulation of biopharmaceutical into the SSUV driven by the pH gradient and also the buffer system to enhance the encapsulation efficiency, preventing the protein aggregation and or degradation.

### FIELD OF INVENTION

The present invention relates to modified compositions of stearically stabilized unilamilar vesicles for encapsulating biopharmaceutical recombinant human proteins. More particularly, the present invention relates to stearically stabilized unilamilar vesicles (hereinafter SSUV) for encapsulating modified pharmaceutical compositions of recombinant human glycoprotein/(human erythropoietin) or its pharmaceutically acceptable derivatives, having long circulating half life with an in-vivo biological activity to increase the production of reticulocytes and red blood cells in intended patients.

### BACKGROUND AND PRIOR ART

Conventionally, certain biopharmaceutical recombinant human proteins have been used for regulating red blood cell production. Erythropoietin or EPO is a glycoprotein produced in the kidney, which regulates red blood cell production by acting on stem cells of the bone marrow. This process is known as erythropoiesis, which occurs to compensate cell destruction and facilitates production of red blood cells for adequate tissue oxygenation. Erythropoeiesis may also occur outside the bone marrow e.g. within the spleen or liver. This is known as extramedullary erythropoiesis.

Biosynthetic manufacturing of erythropoietin using recombinant DNA technology has been known in the art. Recombinant EPO produced by mammalian expression system, preferably CHO cells, where in the gene encoding the human EPO protein is integrated into the chromosome. Recombinant EPO may be therapeutically administered to patients e.g. a recombinant form of human erythropoietin is used as an anti-anemic especially for treatment of anemia in patients with renal failure.

There are certain limitations associated with available therapeutics such as EPO e.g. short plasma half-life and accessibility to protease degradation and hence lower bioavailability and the need for increased frequency of administration to the patients.

Liposomes are vesicles comprising of lipid molecules that have both hydrophilic and hydrophobic parts. Such molecules are known as amphipathic. They are generally arranged in spherical bilayers and can be used to encapsulate various biologically active materials. Liposomes are particularly useful to deliver biologically active materials by encapsulating compounds with limitations, such as lower water solubility. Generally they act as a carrier of the compositions. They carry therapeutic agents to target cells and also act as stabilizers. It is desirable to have an increased circulation time for liposomes, so as to reach the target cell. Liposomes can be used as sustained release systems for biologically active materials. They are well known as drug delivery vehicles. The problem associated with liposomes is destabilization by opsonin protein coating as well as lipoproteins and phospholipases present in body fluid. The rapid clearance of liposome from systemic circulation by reticuloendothelial system has been an important barrier blocking the use of liposome for systemic therapeutic application. Uptake by Mononuclear Phagocyte System (MPS) cells generally leads to irreversible sequestering of encapsulated drug, thereby eliminating any beneficial effects as well as posing potential risk of toxicity to these cells. Phagocytic and endocytic cellular uptake of liposome and its content carried out by macrophage cells of MPS and can occur in all tissues. These cells originate from bone marrow but form a resident population through out the body. Following the intravenous administration, liposome primarily comes in contact with macrophages in liver, spleen and bone marrow where they are removed from circulation. The various interactions of the liposomes are: (1) exchange of materials, primary lipids and proteins with cell membrane, (2) adsorption or binding of liposome to cells, (3) cell internalization of liposomes by endocytosis or phagocytosis once they are bound to cells and (4) fusion of bound liposome to cell membrane. All these interaction depends upon the lipid composition, type of cell, presence of specific receptor and various other features.

Most of the Biotechnology based therapeutic agents, however are not expected to survive macrophage uptake. Results of studies of small peptide hormone in various liposomes showed complete loss of activity, as liposomes are cleared from blood stream.

To overcome this problem with liposomes, various modifications in the processes/methods for liposome preparation have been disclosed in the literature. The liposomes containing high distearylphosphatidyl choline (DSPC) mixed with cholesterol have shown prolonged circulation. In another example preparation of liposome using PEG-phosphatidylethanolamine has proven to offer better protection from MPS uptake and increased blood circulation time.

The U.S. Pat. No. 6,645,522 titled “Erythropoietin liposomal dispersion” assigned to Cilag AG, discloses the liposome composed of PEG-Cholesterol or cholesterol ester in combination with DPPA or DPPG lipid to encapsulate the EPO, using Ethanol injection method. An example provides the use of Cholestrol in combination with DPPA and DPPG. The encapsulation has been carried out by passive diffusion.

The U.S. Pat. No. 4,806,524 titled “Stable erythropoietin preparation and process for formulating the same” assigned to ‘Chugai Seiyaku Kabushiki Kaisha’ proposes a stable erythropoietin preparation containing one or more protein stabilizers and process for formulating the same, wherein liquid formulation as well as lyophilized formulation consisting of human serum abumin, gelatin, mannitol glutathione, glucose, maltose etc as stabilizers.

The U.S. Pat. No. 6,043,094 discloses methods for liposome based therapy, wherein the liposomes have an outer surface that contains an affinity moiety that can affect binding specifically to a biological surface at which therapy is aimed.

The U.S. Pat. No. 6,340,742 discloses a new class of PEG derivatives of EPO, by conjugating the EPO with poly (ethylene glycol), wherein the average molecular weight of each PEG group is about 24 to 35 kilodalton, wherein the PEG group is capped by methoxy group in order to increase the blood circulation time.

The US patent application 2005/0202091 discloses a pharmaceutical composition of erythropoietin that is stabilized with a combination of a Poloxamer polyol and polyhydric alcohol.

The U.S. Pat. No. 7,179,484 discloses a liposome containing lipophillic chemical drug for delivery wherein it has been encapsulated in a liposome coated with protein like albumin to enhance stability.

The coating of these liposomes with emulsifying protein may complicate the analysis of the biopharmaceutical product, like EPO, as, usage of such protein emulsification for delivering the protein based product has great concern, because along with the therapeutically active protein other denatured protein used in emulsification are also injected. Therefore there is a need for vesicles with long circulation time without using the protein emulsifier agent.

### DETAILED DESCRIPTION OF THE INVENTION

As required, detailed embodiments of the present invention are disclosed herein; however, it is to be understood that the disclosed embodiments are merely exemplary of the present invention, which can be embodied in various forms. Therefore, specific structural, functional, formulation or process details disclosed herein are not to be interpreted as limiting, but merely as a representative basis for teaching one skilled in the art to variously employ the present invention in virtually any appropriately detailed structure, process or formulation. Further, the terms and phrases used herein are not intended to be limiting but rather to provide an understandable description of the present invention.

The terms “a” or “an”, as used herein, are defined as one or more than one. The term plurality is defined as two or more than two. The term another, is defined as at least a second or more. The terms including and/or having, as used herein, are defined as comprising (i.e., open language).

An embodiment of the present invention provides pharmaceuticals compositions comprising effective amounts of stearically stabilized unilamilar vesicles containing recombinant human glycoprotein or an erythropoietin moiety of the present invention or pharmaceutically acceptable derivative(s) thereof, having an in-vivo biological activity to increase the production of reticulocytes and red blood cells, with one or more of pharmaceutically acceptable cryopreservative, diluents, emulsifiers, stabilizers, carriers, adjuvants, solubilizers and antioxidants.

An embodiment of the present invention provides stearically stabilized unilamilar vesicles (SSUV) with long circulation time with out using the protein emulsifier agent. In an embodiment, the encapsulation is carried out using the pH gradient leading to better encapsulation efficiency. In an embodiment, PEG-DSPE is used along with other lipids to form the liposome, which has a size of 80-120 nm to from stearically stabilized unilamilar vesicles, which encapsulate the biotherapeutic protein using pH gradient and/or salt gradient in combination of temperature ranges from 4 Degree Celsius to 40 Degree Celsius.

In an embodiment of the present invention, the surface coating to form SSUV provided by the hydrophilic polymer chains provides colloidal stability and serves to protect the SSUV loaded with an effective amount of human recombinant protein or glycoprotein preferably, erythropoietin, and increases the sustained release effect of such SSUVs by decreasing or protecting the SSUV from uptake by reticuloendothelial system, thus providing several fold long blood circulation lifetime of SSUV-EPO in comparison to normal EPO. The embodiment of present invention of SSUV-EPO, wherein these SSUV prevents the self aggregation of the liposome with liposomes, resulting in increased blood circulation time and also a dysopsonization phenomenon where PEG actually promotes binding of certain proteins that, then, masks the vesicle and prevents their clearance by reticuloendothelial system. In an embodiment of the present invention, the pharmaceutical composition comprises various lipids, covalently modified lipids with polyethylene glycol and/or neutral detergent to form long circulating and tightly packed lipid vesicles. The composition reduces the reticulo-endothelial clearance of SSUV and causes the sustained released effect of encapsulated biopharmaceutical and recombinant human glycoprotein or an erythropoietin moiety. PEG-SSUV are unilamilar lipid vesicles of uniform size in the range of 50-300 nm and is a modified form of conventional liposome. This can overcome the problem of destabilization because of opsonin protein coating as well as due to lipoproteins and phospholipases present in body fluid. Rapid clearance of conventional liposome from systemic circulation by reticuloendothelial system has been an important barrier blocking the use of liposome for systemic therapeutic application. The composition of the lipid makes the SSUV more rigid, reduces the passive leakage and also the degradation of SSUV.

An embodiment of the present invention provides a modified composition of a vesicle containing a biopharmaceutical recombinant human protein having an in-vivo biological activity to increase the production of reticulocytes and red blood cells. An exemplary embodiment of the present invention provides a method for formulating the biomolecule with enhanced blood circulation time of EPO and reduced clearance of liposome by reticuloendothelial system by encapsulating the human recombinant protein, glycoprotein, preferably human EPO, in stearically stabilized unilamilar vesicles (SSUV), which is composed of hydrophilic polymer like polyethylene glycol chains having molecular weight in the range of 350 to 12,000 daltons, more preferably 350-5000 daltons molecular weight, covalently attached to distearoylphosphatidylethanolamine (DSPE) or PE phospholipid. In an embodiment of the present invention, DSPE may bear the multiarm PEG of 2000 dalton molecular weight. In an embodiment, the hydrophilic polymer forming the coating around the liposome in non continuous manner is selected from group of ploymethylacrylamide, polyethylene glycol, polypropylene glycol, polyvinylpyrrolidove, GM1 ganglioside, preferably polyethylene glycol. In the preferred embodiment, the moiety is bound directly to surface lipid component by covalent attachment to the head group of a vesicle forming phospholipid.

In an embodiment of the present invention, the SSUV composition comprises of the mPEG-DSPE, Hydrogenated Soya Phosphatidyl Choline, and Cholestrol, where in the mPEG have molecular weight of 350-5000 daltons. In an embodiment, the DSPE may also contain the multiarm mPEG of 2000 molecular weight.

In another embodiment of the present invention, the SSUV composition comprises of the mPEG-DSPE, Hydrogenated Soya Phosphatidyl Choline, Cholestrol and Polysorbate-20 or 80. In an embodiment of the present invention, ‘polysorbate 80’, which is nonionic surfactant, is integrated into lipid vesicle. This arrangement facilitates the encapsulation with enhanced blood circulation.

In a yet another embodiment of the present invention the SSUV comprises mPEG-DSPE and mPEG-1,2-distearoyl-sn-glycero-3-phosphocholine (DSPC) or DSPC along with Hydrogenated Soya Phosphatidyl Choline, Cholestrol and/or molecules like hydrophilic polyethylene-polyoxypropylene block copolymer molecular weight ranging between about 2000 to 15000 daltons. In an embodiment the preferred grades includes poloxamer 188 (PLURONIC F68™), Poloxamer 237 (PLURONIC F87), Poloxamer 338 (PLURONIC F108), Poloxamer 407 (PLURONIC F127), and/or nonionic surfactants to further increase the sturdiness of liposome by DSPC incorporation and mPEG will contribute stearically stabilized properties of SSUV, this will further enhance the blood circulation and cause the sustained release impact of EPO, when encapsulated.

An embodiment of the present invention, provides a composition comprising stabilizing agent and or pharmaceutically acceptable excipients selected from for example, amino acid like L-methionine, L-arginine, Glycine, Histidine, Alanine and salt thereof, Suger like, sucrose, mannose, trehalose, mannitol, glycerol and benzyl alcohol, or any combination thereof.

An embodiment of the present invention provides a method for formulating a biomolecule with enhanced blood circulation time and reduced clearance of liposome by reticuloendothelial system by encapsulating the human recombinant protein, glycoprotein, preferably human EPO, in stearically stabilized unilamilar vesicles (SSUV), which is composed of hydrophilic polymer like polyethylene glycol chains having molecular weight in the 350 to 12,000 daltons, more preferably 750, 2000 and or 5000 daltons molecular weight, covalently attached to distearoylphosphatidylethanolamine (DSPE) or PE phospholipid. In an embodiment, DSPE may bear the multiarm PEG of 2000 dalton molecular weight. The hydrophilic polymer forming the coating around the liposome in non-continuous manner is selected from a group of polymethylacrylamide, polyethylene glycol, polypropylene glycol, polyvinylpyrrolidove, GM1 ganglioside, preferably polyethylene glycol. In an embodiment, the moiety is bound directly to surface lipid component by covalent attachment to the head group of a vesicle forming phospholipid. In an embodiment of the present invention, the method comprises selecting a predetermined combination(s) of lipids, dissolving the predetermined combination of the lipids in suitable organic solvent(s). In an embodiment, the process comprises, hydrating the lipid layer by using suitable aqueous buffer, like citrate buffer, phosphate buffer, preferably kosmotropes especially ammonium sulfate containing buffer with or with out biopharmaceutical stabilizing agent and or pharmaceutically acceptable excipients like amino acid such as L-methionine, L-arginine, Glycine, Histidine, Alanine and salt thereof, Sugar like, sucrose, mannose, trehalose, mannitol, glycerol and benzyl alcohol, or any combination thereof. In an embodiment, the process comprises homogenizing and extruding to achieve required particle size of PEG-SSUV. In an embodiment the required particle size is in the range of 50-300 nm. An embodiment of the present invention provides a process of encapsulation of biopharmaceutical, which will be driven by pH gradient using 3.0-8.5 pH, preferably 3.5-7.5 pH, where in the liposome has internal pH of 3.0-5.5 pH more preferably 4.0-5.5 pH and immediate environment has a pH of 5.6-8.5, preferably, 6-7.5 pH and/or salt gradient with or with out pharmaceutical stabilizing agent and/or pharmaceutically acceptable excipients like amino acid like L-methionine, L-arginine, Glycine, Histidine, Alanine and salt thereof, Sugar like, sucrose, mannose, trehalose, mannitol, glycerol and benzyl alcohol, in any combination thereof.

An embodiment of the present invention provides a process of encapsulation of biopharmaceutical (EPO), which will be driven by a salt gradient using 10 mM-1000 mM, preferably 10-300 mM with or without biopharmacetical stabilizing agent and or pharmaceutically acceptable excipients like amino acid like L-methionine, L-arginine, Glycine, Histidine, Alanine and salt thereof, sugar like, sucrose, mannose, trehalose, mannitol, glycerol and benzyl alcohol, in any combination thereof.

In another embodiment of the present invention, the process further comprises of re-hydration process of SSUV carried out using suitable aqueous buffer, like citrate buffer, phosphate buffer, preferably kosmotropes especially ammonium sulfate containing buffer.

An embodiment of the present invention provides a method of treating blood disorders such as defective red blood cell production, low red blood cell production comprising administering a therapeutically effective amount of the modified composition of the present invention. An exemplary embodiment of the present invention provides a method of treating anemia comprising administering a therapeutically effective amount of a suitable biopharmaceutical compound using SSUV of the present invention. The therapeutically effective amount may be determined as the amount necessary for the in vivo activity to cause bone marrow cells to increase production of reticulocytes and red blood cells. This amount may vary depending upon factors such as the type and extent of problematic condition being treated, the condition of the patient and the primary cause of the anemia. The frequency of administration may be reduced by using the therapeutically effective amount of the composition of the present invention. The dosing frequency may also vary due to difference in response to the dose by different patients.

The various embodiments of the present invention provide PEG-SSUV compositions with increased circulation half-life and stability and also the desirable biological activity. These PEG-SSUV encapsulate biotherapeutic protein, Glycoprotein, or more specifically EPO enables it to over come the problem of short serum half-life and increases the efficacy of the EPO and its plasma residence time. Wherein these PEG-SSUV-EPO compositions prevent the self aggregation of the liposome with liposomes, resulting in increased blood circulation time and also a dysopsonization phenomenon where PEG actually promotes binding of certain proteins that, then, masks the vesicle and prevents their clearance by reticuloendothelial system.

The activity of modified composition of the present invention can be determined by known methods (e.g. normocythaemic mouse assay). The composition of the present invention can be administered in a therapeutically effective amount to patients at a relatively lower frequency and/or dosage. The compositions/formulations of the present invention may be presented in unit dosage form and may be prepared by any of the method well known in art. The suitable formulations include the aqueous sterile injection solution, which may contain antioxidants, buffers, bacteriostates and solutes which render the formation isotonic with the body fluid especially blood of the patient. In an embodiment of the present invention, the formulation may be prepared in unit-dose or multiple doses in pre-filled injection, ampoules or vials and may be in lyophilized form.

In the foregoing disclosure, exemplary embodiments of the invention have been described. However, one of ordinary skill in the art appreciates that various modifications and changes can be made without departing from the scope of the invention. Accordingly, the provisional specification is to be regarded in an illustrative rather than a restrictive sense, and all such modifications are intended to be included within the scope of the invention. The benefits, advantages, solutions to problems, and any element(s)/feature(s)/step(s) that may cause any benefit, advantage, or solution to occur or become more pronounced are not to be construed as a critical, required, or essential features or elements with reference to the disclosure.

### Example 1

### Preparation of the Pegylated Liposome by Hydration Method

Lipid Film Preparation: The organic mixture of chloroform and methanol was prepared and N-(Carbonyl-methoxypolyethylene glycol 2000)-1,2-disteroyl-sn-glycero-3-phosphoethanolamine sodium salt (7-10 milligram per milliliter), Hydrogenated soy phosphatidylcholine (2 to 4 milligram per milliliter), and Cholesterol (2 to 4 milligram per milliliter) were dissolved one after another in organic mixture in a inlet flask. In another composition the lipid content was reduced to thirty percent. They were mixed to form the clear solution of the lipid. The inlet temperature was adjusted to 60-65° C. and outlet temperature was set to 45° C. The solvent was evaporated by spray drying at 20-40 ml per minutes and thin lipid film around the wall of the flask was collected. The flask was flushed with nitrogen to dry off any residual solvent. Check the content in spray dry powder. Hydration of lipid film: The lipid film was hydrated using lipid and drug ration of 1:100 to 1:250 in 40 ml of aqueous hydration media containing 0.15 molar ammonium sulphate solution pH-4.80. The solution was sonicated at 60° C. for one hour. Homogenization of hydrated lipid solution was carried out in homogenizer at 60° C. and 12000 psi, 2 to 10 passes. After homogenization particle size and pH were measured. The sephadex G50 resin was swelled in histidine sodium citrate buffer pH-7.20 and packed in column and liposome buffer exchange was carried out using histidine sodium citrate buffer pH-7.20.

The liposomal suspension was further processed for size selection by extruding successively through filter having pore size from 1.0 μm, 0.4 μm and 0.05 μm.

Passive Protein Encapsulation: In clean glass bottles, take 15 ml of liposome solution and add the erythropoietin protein from 5000 IU-30000 IU per ml of solution and incubate it up to 48-72 hours at various temperatures like 10° C., 30° C., 37° C. and 52° C. in water bath. The pegylated liposomal EPO (PEG-SSUV-EPO) composition thus obtained was then aseptically filtered using a sterile 0.22 micron meter filter into a sterile depyrogenated container. Withdraw 1.0 ml of the samples for encapsulation analysis and keep it at 2-8° C. until used, and analyze for the following parameters:
1. Appearance: White opaque colored translucent liquid.
2. pH: 7.2±0.2
3. Particle size: Average particle size: 100-120 nm
4. Encapsulation Efficiency: At temperature 10-37° C. is >56%, whereas the encapsulation efficiency at temperature 52° C. is only 2-4%, which is due to the protein aggregation at high temperature which leads to decreased encapsulation and also change in the lipid vesicle integrity.

By decreasing the hydration media or increasing the concentration of the various mentioned phospholipids, the resulting pegylated liposome have an increase phospholipids content per unit area. This increased content increases the liposome stability, decreases permeability, and creates the sustained release effects. We have made balance between the liposome stability and entrapment of the protein molecule by adjusting the concentration and ratio, which give rise to improved passive encapsulation of protein in pegylated liposome.

### Example 2

### Preparation of the Liposome by Reverse Phase Method

The organic mixture of chloroform and methanol was prepared and N-(Carbonyl-methoxypolyethylene glycol 2000)-1,2-disteroyl-sn-glycero-3-phosphoethanolamine sodium salt (7-10 milligram per milliliter), Hydrogenated soy phosphatidylcholine (2 to 4 milligram per milliliter), Cholesterol (2 to 4 milligram per milliliter) and Ethanolamine phosphoglycerides (2 to 4 milligram per milliliter) were dissolved, one after another in organic mixture of chloroform and methanol in 1:1 ratio in a inlet flask. The content were mixed continuously until the clear solution is formed. The organic content of the mixer was evaporated by rotary evaporation at low temperature and under vacuum, resulting the formation of thin film of lipids around the walls of the flask. After releasing the vacuum the flask was rotated for few minutes while passing the dry nitrogen into the flask to dry off any residual solvent. The lipid film was rehydrated in 40 ml of aqueous hydration media containing histidine sodium citrate buffer containing 5000 to 30,000 IU/ml of erythropoietin. The mixture was homogenized for several passes at slow speed to avoid the protein degradation or denaturation. The size selection of the pegylated liposomal solution obtained from the above process was carried out by extruding successively through filter having pore size of 1.0 μm, 0.4 μm, 0.2 μm, and 0.1 μm in sequential pass. The pegylated liposomal EPO (PEG-SSUV-EPO) solution was further incubated at different temperature for the passive encapsulation to increase the encapsulation efficiency of the process. The sterile filling of PEG-SSUV-EPO was carried out at various dosage form with nitrogen purging. The samples were analyzed for various quality control parameters for batch release like, appearance, pH, In-vitro activity, In-vivo potency, lipid: protein ratio, sterility, safety toxicity, PK/PD, in-vitro serum stability etc.

### Example 3

### Preparation of the Liposome by Modified Reverse Phase Method

In another embodiment, the organic mixture of Methanol:Ethanol was prepared and N-(Carbonyl-methoxypolyethylene glycol 2000)-1,2-disteroyl-sn-glycero-3-phosphoethanolamine sodium salt (7-10 milligram per milliliter), Hydrogenated soy phosphatidylcholine (2 to 4 milligram per milliliter), and Cholesterol (2 to 4 milligram per milliliter) and Ethanolamine phosphoglycerides and/or Egg lecithin (2 to 4 milligram per milliliter) were dissolved one after another in organic mixture in a inlet flask and drop wise added into the small volume of i.e. one forth histidine containing sodium citrate buffer was than added. The content were sonicated at 35-40° C. using pulse process of sonication. The above mixture was drop by drop added to the another lot of aqueous hydration media like histidine sodium citrate buffer containing 5,000 to 30,000 IU/ml of erythropoietin with continuous mixing and gentle agitation. The lipid content can directly be added to the aqueous media hydration media like histidine sodium citrate buffer containing 5,000 to 30,000 IU/ml of erythropoietin drop wise. This process will reduce the denaturation of protein at greater extent. The mixture was homogenized for several passes at low speed to avoid the protein degradation or denaturation but to obtain the appropriate liposomal size. The size selection of the pegylated liposomal EPO (PEG-SSUV-EPO) solution obtained from the above step was carried out by extruding successively through filter having pore size from 1.0 μm, 0.4 μm, 0.2 μm and 0.1 μm. The pegylated liposomal EPO solution was further incubated at different temperature for the passive encapsulation to further increase the entrapment efficiency of the process. The samples were analyzed for various parameters for quality control testing for batch release like, appearance, pH, In-vitro activity, In-vivo potency, lipid: protein ratio, sterility, safety toxicity, PK/PD, in-vitro serum stability etc.

### Example 4

### Encapsulation Efficiency Determination

Ion exchange chromatography and RPHLC were used to determine the amount of protein associated with lipids in solution. Ion exchange chromatography used for the separation of free EPO protein from lipid associated EPO protein. Strong anion resin supplied in pre-swollen in 20% ethanol and slurry was prepared by decanting the 20% ethanol solution and replace it with starting buffer in a ratio of 1:3. All the material was equilibrated to the temperature at which the chromatography was performed. The gel slurry was degassed. The column has been equilibrated with buffers at reduced flow rates after packing is completed. The resin was equilibrated with ten column volumes of 50-100 milli molar Tris buffer (pH 8.0) at gravitational flow. The pH of the flow through was monitored to ensure proper equilibration. The EPO protein was diluted fifty to hundred times in equilibration buffer and loaded on the column. The flow through was collected in small fraction. The column was washed with three to ten column volume of washing buffer. The elution was carried out using elution buffer containing 50-100 milli molar Tris buffer pH 8.0 containing one molar sodium chloride. The sample consists of purified EPO protein, EPO protein spiked in blank pegylated liposomal solution as positive control and pegylated liposomal EPO formulation of various compositions. After elution the resin was regenerated using two-column volume of 0.2N sodium hydroxide solution followed by five-ten column volume of water and equilibration of resin with ten-column volume using Tris Buffer. The over all process efficiency is 80-95%, wherein the spiked EPO in the blank liposome has been able to bind to resin greater than 97-98%.

The flow through fraction containing pegylated liposome-EPO was solubilized in 0.1% of 50-100 milli molar hydrochloric acid containing isopropyl alcohol in ratio of 1:2 and extracted with chloroform. The mixture was vortexed for thirty seconds and centrifuged at 6000 revolution per minutes for 1-2 minutes. The upper aqueous layer containing extracted EPO was separated carefully to avoid the mixing of the interphase. The process recovery was 80-85%. To increase the efficiency of the analytical process direct injection method has been established, wherein the flow through fraction containing pegylated liposomal EPO was injected into column and on column lysis and detection carried out on reverse phase C18 column (Supelco, 5μ, particle size, 5 cm×4.6 mm). Amount of EPO was determined by comparing the area obtained from principle peak of EPO protein with established calibration curve of EPO ranging from 5 μg/ml to 50 μg/ml with 95-98% accuracy. Mobile phase A (water+0.1% TFA) and B (Acetonitrile+0.1% TFA) was used for separation of protein from lipids. A linear gradient of 6.66% per minute was used between 40 to 60% solvent B at flow rate of 1-2 ml/min and column temperature at 55-60° C. Direct injection was preferred as initial gradient was sufficient to dissolve complete lipid and there is no possibility of EPO protein to elute in void volume. The detection of protein was carried out at 210 nm using UV detector. The data was used for determining the % association or encapsulation or entrapment of EPO with pegylated liposome. To determine the protein vs lipid ratio in given pegylated liposomal composition, Lipid content for eg cholesterol, DSPE, HSPC, was estimated in pre and post column processing. A reverse phase C18 column (ABZ+PLUS, 5μ particle size, 25 cm×4.6 mm) was used for quantitation of different lipids. Mobile phase A (water+0.15% TFA) and B (Acetonitrile/Isopropanol+0.05% TFA) was used for separation with a linear gradient of 5% per minute between 50% to 100% of solvent B at flow rate of 0.5 ml/min. Detection of lipid was carried out at 205 nm using UV detector. The standard calibration curves (5 μg/ml to 100 μg/ml) were prepared by using Cholesterol and HSPC lipid. The amount of lipid in each fraction was estimated using calibration curve and represented in mole ratio. The encapsulation efficiency was determined by ratio of [protein/lipid] after chromatography and before chromatography. The data of passive encapsulation process showed an efficiency of >56% for samples incubated at 10 and 30° C. temperature. But the encapsulation efficiency for 52° C. temperature samples was found drastically reduced to 4.12% indicating that high temperature causes the protein aggregation.

### Example 5

### In-Vivo Pharmacodynamics in Rat

Eight week-old wistar rats (about 200-300 g) had been used for the study and were procured from CPSCEA certified supplier. The animals were acclimatized for 3-4 weeks in the animal house at 21° C. temperature and 53-55% relative humidity. The study groups comprised of EPO, PEG-SSUV-EPO (pegylated liposomal EPO preparation) and vehicle control (PBSA). The animals had administered (200 IU/200 g) of the test samples, EPO, PEG-SSUV-EPO (pegylated liposomal EPO preparation) prepared by hydrataion and rehydration method with encapsulation at 10° C. and PBSA as vehicle control through subcutaneous route, the animals returned to cages for further blood collections. Blood was collected from the retro orbital plexus region before dosing and on 1, 2, 3, 4, 5, 6, 7, 8, 9, 10, 11, 12, 13, 14, 15, and 16^{th} days after administration of drug. The blood samples were used for reticulocyte analysis with in 48 hours of collection. For analysis 2 μL of well-mixed whole blood was added in 1.0 ml of PBS and 1.0 ml of retic reagent. Reticulocyte count as a percentage of total erythrocytes in mice peripheral blood was estimated using EpicXL Flow Cytometer. The percentage of reticulocytes is determined using a biparametric histogram: number of cells/red fluorescence (620 nm). Equation for percent (%) positive reticulocyte {(%) Gated stained tube−(%) gated unstained tube=(%) reticulocytes}. The data summarized and plotted using graph pad prism software having days on x-axis vs percentage reticulocyte on y-axis and (AUC) area under curve was determined. It was found that PEG-SSUV EPO ie pegylated liposomal EPO formulation showed 71% more AUC for reticulocyte production over a period of time in comparison to EPO in rat.

### Example 6

### Stability of the Pegylated Liposomal EPO Formulation

The above-mentioned formulation was kept at 2-8° C. for three months and samples were processed as described in example 3. The percentage encapsulation efficiency retained in stored solution is between 92-95 of its original encapsulation or association efficiency.

### Example 7

### Safety Toxicity

The acute toxicity studies were conducted in small rodent animals, for example in rat and mice by administering the pegylated liposomal EPO solution by s.c. and i.v. routes in a single dose of 4000 IU/kg body weight. The animals were observed for fifteen days. On day fifteenth the animals were dissected for gross anatomy examination. There was no death or any abnormality found in the gross organ examination in all the species.

### DESCRIPTION OF FIGURES

FIG. 1: Process out line for preparation of Pegylated liposomal EPO injection by various methods.

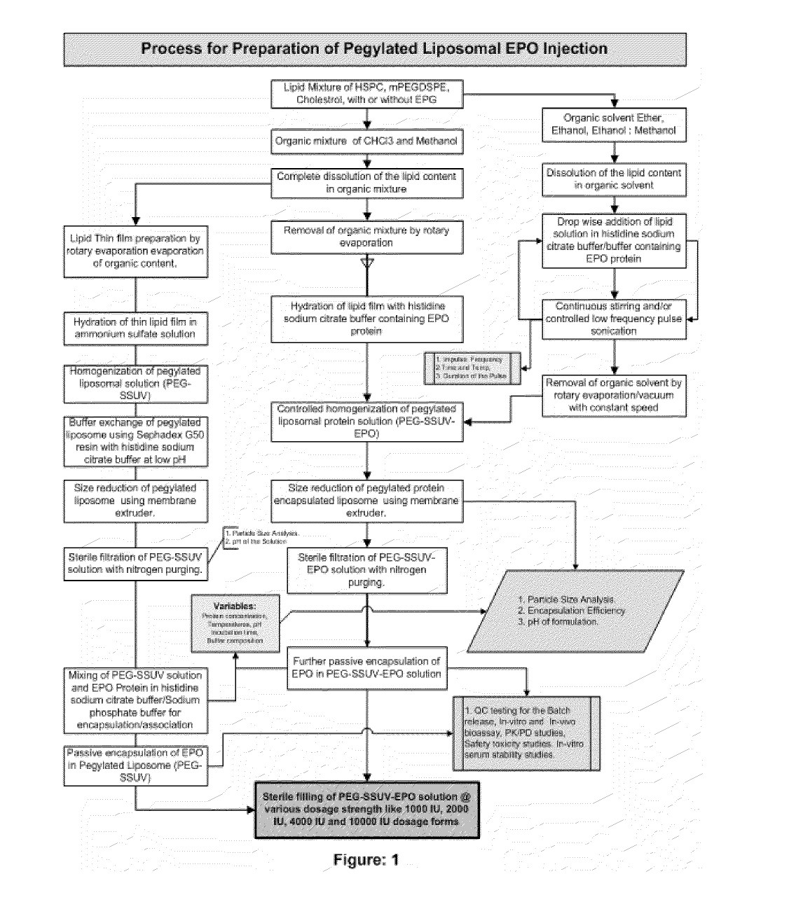

FIG. 2: Pictorial Depiction of protein encapsulated, associated pegylated liposome

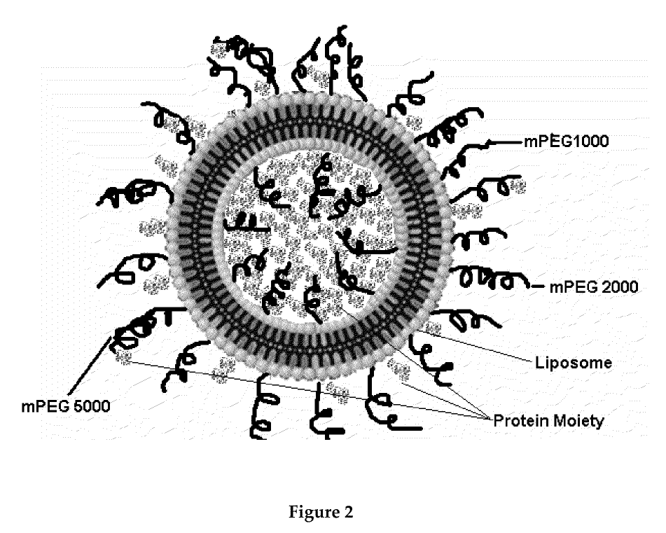

### REFERENCES

Alkan-Onyuksel, H. et al., “A Mixed Micellar Formulation Suitable for the Parenteral Administration of Taxol,” Pharmaceutical Research, 11(2):206-212 (1994).
Allen, T. M. et al., “Large Unilamellar Liposomes with Low Uptake into the Reticuloendothelial System,” Federation European Biochemical Societies, 223(1):42-46 (October, 1987).
Allen, T. M. et al., “Liposomes containing synthetic lipid derivatives of poly(ethylene glycol) show prolonged circulation half-lives in vivo,” Biochimica et Biophysica Acta, 1066:29-36 (1991).
Beaumier, P. L. et al., “Effect of Liposome Dose on the Elimination of Small Unilamellar Sphingomyelin/Cholesterol Vesicles from the Circulation,” Research Communications in Chemical Pathology and Pharmacology, 39(2):277-289 (February, 1983).
Bedu-Addo, F. K. et al., “Interaction of Polyethyleneglycol-Phospholipid Conjugates with Cholesterol-Phosphatidylcholine Mixtures: Sterically Stabilized Liposome Formulations,” Pharmaceutical Research, 13(5):718-724 (1996).
Campbell CD, “Liposome based formulation with Protein and sustained release effect”, Biochim Biophys Acta, 730(1): 95-103 (1983).
C. Carrion, “Preparation of Long-Circulating immunoliposomes using PEG-Cholesterol conjugates: effect of the spacer arm between PEG and cholesterol on Liposome Characteristics”, Chemistry and Physics of Lipids 113: 97-110 (2001).
Corine C. Visser, “Targeting Liposomes with protein drugs to the blood-brain barrier in-Vitro”, European Journal of Pharmaceutical Sciences 25: 299-305 (2005).
D. Papahadjopoulos “Steriaclly stabilized liposomes: Improvements in pharmacokinetic and anti tumor therapeutic efficacy”, Proc. Natl. Acad. Sci USA, Vol. 88: 11460-11464(Dec. 1991).
D. D. Lasic, “Sterically stabilized liposomes: a hypothesis on the molecular origin of the extended circulation times”, Eochimica et Biophysica 1070: 187-192 (1991)
Denielle N. McLennan, “Lymphatic Absorption is the Primary Contributor to the Systemic Availability of Epoetin Alfa Following Subcutaneous administration to sheep”, The Journal of Pharmacology and Experimental Therapeutics, Vol 313 (1): 345-351 (2005)
Dinesh Shenoy, “Poly (Ethylene Oxide)-Modified Poly (Beta-Amino Ester) Nanoparticles as a pH-sensitive system for Tumor-Targeted Delivery of hydrophilic Drugs: Part2. In-Vivo Distribution and Ti\umor Localization Studies”, Pharma Res. 22 (12): 2107-2114 (Dec. 2005).
Deborah A. Eppstein, “Altered Pharmacological Properties of Liposome-Associated Human Interferon-Alpha”, Journal of Virology: 575-582 (Feb. 1982).
E. C. M. Cabral, “Preparation and Characterization of Liposomes entrapping Allergenics Proteins” Brazilian Journal of Chemical Engineering, Vol. 21(02): 137-146 (April-June 2004).
Eli Kedar, “Delivery of Cytokines by Liposomes.3.Liposomes Encapsulated GM-CSF and TNF-alpha shows improved Pharmacokinetic and Biological Activity and reduced Toxicity in Mice”, Journal of Immunotherapy 20(3): 180-193 (1997).
Emma Johansson, “Development and initial evaluation of PEG-Stabilized bilayer disks as novel model membranes”, Biophysical Chemistry 113:183-192 (2005).
F. Gilbert, “Pharmacokinetics and effect of recombinant human EPO after IV and SC Injection in Healthy Volunteers”, Blood Vol 76(9): 1718-1722 (1990).
Gabizon, A. et al., “Liposome Formulations with Prolonged Circulation Time in Blood and Enhanced Uptake by Tumors,” Proc. Natl. Acad. Sci. (USA), 85:6949-6953 (September, 1988).
Gregoriadis, G. et al., “Liposomes in Drug Delivery: Clinical, Diagnostic and Ophthalmic Potential,” Drugs, 45(1):15-28 (1993).
Haghjoo, K. et al., “Solution Structure of Vasoactive Intestinal Polypeptide (11-28)-NH.sub.2, a Fragment with Analgesic Properties,” Peptide Resesarch, 9(6):327-331 (1996).
Howard J. Lim, “Role of drug release and liposome-mediated drug delivery in governing the therapeutic activity of liposome mitoxantrone used to treat Human A431 and LS 180 solid tumors”, The journal of Pharmacogy and Experimental Therapeutics, Vol. 292 (1): 337-345 (2000).
Hristova, K. et al., “Effect of Bilayer Composition on the Phase Behavior of Liposomal Suspensions Containing Poly(ethylene glycol)-Lipids,” Macromolecules, 28(23):7793-7799 (1995).
Hwang, K. J., “Liposome Pharmacokinetics,” in Liposomes from Biophysics to Therapeutics, Ostro, M. J. (Ed.), Marcel Dekker, Inc., New York, pp. 109-156 (1987).
Heinz Ludwig, “rHuEPO and treatment Outcomes the Preclinical Experience”, The Oncologist 9(5): 48-54 (2004).
Iain C. Macdougall, “Pharmacokinetics and Pharmacodynamics of Intravenous and Subcutaneous EPO Receptor Activator (C.E.R.A) in Patients with Chronic Kidndey Disease”, American Aociety of Nephrology, 1211-1215 (2006)
Iain C. Macdougall, “Pharmacokinetics of Novel Erythropoiesis Stimulating Protein Compared with Epoetin Alfa in Dialysis Patients”, Journal of American Society of Nephrology 10:2392-2395 (1999).
Iain C. Macdougall, “Pharmacokinetics and Pharmacodynamics of IV and Sc Continuous EPO receptor Activator (C.E.R.A) in patients with Chronic Kidney Disease”, American Society of Nephrology 1:1211-1215 (2006).
Iain C. Macdougall, “Pharmacokinetics of novel Erythropoiesis Stimulating Protein Compared with Epotin Alfa in Dialysis Patients”, Journal of American Society of Nephrology 10:2392-2395 (1999).
Jian QIU. “Multivesicular Liposome Formulations for the sustained delivery of interferon alpha-2b”, Acta Pharmacologica Sinica, 26 (11), 1395-1401 (2005)
Jacques-Philippe Colletier, “Protein encapsulation in liposomes: efficiency depends on interaction between protein and phospholipid bilayer”, BMC Biotechnology, 1-8(10.05.2002).
Jorge F Perez-Oliva, “Comparison of two recombinant EPO formulations inb patients with anemia due to end stage renal disease on hemodialysis: A parallel, randomized, double blind study”, BMC Nephrology: 1-11(May 23, 2005).
Ka-Yun Ng, “The effect of Polyethylene glycol (PEG)-derived lipid on the activity immuno lipo some”, International Journal of Pharmaceutics 193: 157-166 (2000).
Martin C. Woodle, “Controlling Liposome blood clearance by surface-grafted polymers”, Advanced Drug Delivery Review32: 139-152 (1998).
Koen Jolling, “Mexed-Effect modelling of the interspecies pharmacokinetic scaling of Pegylated human EPO”, European Jourmnal of Pharmaceutical Sciences 24: 465-475 (2005).
Koen Jolling, “Population Pharmacokinetic Analysis of Pegylated Human EPO in Rats,” Journal of Pharmaceutical Sciences, Vol-93(12):3027-3037 (2004).
Kaiser, E. T. et al., “Peptides with Affinity for Membranes,” Ann. Rev. Biophys. Biophysical Chem., 16:561-581 (1987).
Kedar, E. et al., “Delivery of Cytokines by Liposomes. I. Preparation and Characterization of Interleukin-2 Encapsulated in Long-Circulating Sterically Stabilized Liposomes,” Journal of Immunotherapy, 16:47-59 (1994).
Kenworthy, A. K. et al., “Structure and Phase Behavior of Lipid Suspensions Containing Phospholipids with Covalently Attached Poly(ethylene glycol),” Biophysical Journal, 68:1903-1920 (May, 1995).
Kirby, C. et al., “Dehydration-Rehydration Vesicles: A Simple Method for High Yield Drug Entrapment in Liposomes,” Biotechnology, pp. 979-984 (November 1984).
Kirby, C. et al., “Effect of the Cholesterol Content of Small Unilamellar Liposomes on their Stability in vivo and in vitro,” Biochem. J., 186:591-598 (1980).
Klibanov, A. L. et al., “Activity of amphipathic poly(ethylene glycol) 5000 tp prolong the circulation time of liposomes depends on the liposome size and is unfavorable for immunoliposome binding to target,” Biochimica Biophysica Acta, 1062:142-148 (1991).
Klibanov, A. L. et al., “Amphipathic polyethyleneglycols effectively prolong the circulation time of liposomes,” FEBS Lett., 268(1):235-237 (July, 1990).
Lasic, D. et al., Stealth Liposomes, Lasic, D. et al., (Eds.), CRC Press, Inc., Boca Raton, Fla., pp. 1-289 (1995).
Litzinger, D. C. et al., “Effect of Liposome Size on the Circulation Time and Intraorgan Distribution of Amphipathic Poly(ethylene glycol)-Containing Liposomes,” Biochimica et Biophysica Acta, 1190:99-107 (1994).
Liu, D. et al., “Role of liposome size and RES blockade in controlling biodistribution and tumor uptake of GM.sub.1-containing liposomes,” Biochimica et Biophysica Acta, 1104:95-101 (1992).
Li Yang, “A novel method to prepare highly encapsulated interferon-alpha-2b cointaining Liposomes for intramuscular sustained release”, European Journal of pharmaceutics and Bippharmaceutics 64: 9-15, (2006)
Lionel D. Lewis, “Preclinical and Clinical Studies: A Preview of Potential Future Applications Of Erythropoietic Agents”, Elswvier seminars in Hematology, 1-9 (2004)
“M. L. Van Slooten,” “Liposomes as sustained release system for human interferon-gamma: Biopharmaceutiacal aspects” “, Biochimica et Biophysica Acta 1530: 134-145 (2001).”
Martin C. Woodle, “Sterically Stabilized Liposome tjerapeutics”, Advanced Drug Delivery Reviews, 16: 249-265 (1995).
Martin C. Woodle, “Surface Modified liposomes: assessment and characterization for increased stability and prolonged blood circulation”, Chemistry and Physics of Lipids. 64:249-262 (1993).
Michael J. Parnham, “Non Clinical safety studies on Biosimilar Recombinant Human EPO”, Nordic Pharmacological Society Basic & Clinical Pharmacology & Toxicology, 100: 73-83 (2007)
Moshe Mittelman, “EPO induces tumor regression and antitumor immune responses in murine myeloma models”, PNSA, Vol 98(9):5181-5186 (2001).
Monia Zignani, “IN-Vitro Characterization of a novel polymeric-based pH-sensitive Liposome system”, Biochimica et Biophysica Acta 1463: 383-394 (2000)
Motohiro Kato, “Mechanism for the Nonlinear Pharmacokinetics of EPO in Rats”, The Journals of Pharmacology and Experimental Therapeutics, Vol 283(2): 520-527 (1997).
Motohiro Kato, “Pharmacokinetics and Pharmacodynamics of Recombinant Human EPO in Rats, Biotechnology in Drug Research, 51(1):91-95 (2001).
Mysore P. Ramprasad, “Sustained-delivery of an apolipoproteinE-Peptidomimetics using multivesicullar liposomes lowers serum cholesterol levels”, Journal of controlled release 79: 207-218 (2002).
MacDonald, R. C. et al., “Small-Volume Extrusion Apparatus for Preparation of Large, Unilamellar Vesicles,” Biochimica et Biophysica Acta, 1061:297-303 (1991).
Maruyama, K. et al., “Effect of Molecular Weight in Amphipathic Polyehtyleneglycol on Prolonging the Circulation Time of Large Unilamellar Liposomes,” Chem. Pharm. Bull., 39(6):1620-1622 (1991).
Nucci, M. L. et al., “The Therapeutic Value of Poly(ethylene glycol)-Modified Proteins,” Advanced Drug Delivery Reviews, 6:133-151 (1991).
P. Veng-Pedersen, “An Integrated Pharmacodynamics Analysis of EPO, Reticulocyte and Hemoglobin response in Acute Anemia”, Pharmaceutical Research Vol. 19(11):1630-1635 (2002)
Paola Crosasso, “Praparation, Characterization and properties of Srerically Stabilzed paclitaxel-Containing Liposomes”, Journal of Controlled Release 63:19-30 (2000)
Peter Layerman, “Recognition and clearance of methoxypoly(ethyleneglycol)2000-grafted liposomes by macrophages with enhanced phagocytic capacity Implications in experimental and clinical oncology”, Biochimica et Biophysica Acta 1526:227-229 (2001).
Peter Layerman, “Factors affecting the Accelerated Blood Clearance of Polyethylene Glycol-Liposomes upon Repeated Injection”, The journal of Pharmacology and Experimental Therapeutics, Vol. 298 (2):607-612 (2001)
Pierrot Harvie, “Comparative Pharmacokinetics, Distribution in Tissue, and Interactions with Blood Proteins of Conventional and Sterically Stabilized Liposomes Containing 2′,3′-Dideoxyinosine”, Antimicrobial Agent and Chemotherapy. Vol. 40(1): 225-229 (1996).
P. Lemieux, “A combination of poloxamers increases gene expression of plasmid DNA in skeletal muscle', Gene Therapy 7: 986-991 (2000).
Ralf Ignatius, “Presentation of proteins encapsulated in steriaclly stabilized liposomes by dendritic cells initiates CD8+ T-cell responses in-Vivo”, Blood, Vol: 96: 3505-3513 (November 2000).
Rohini Ramakrishnan, “Pharmacokinetics and Pharmacodynamics modeling of Recombinant Human Epo after IV and SC dose administration in Cynomolgus Monkeys”, The Journals of Pharmacology and Experimental Therapeutics, Vol 306(1): 324-331 (2003)
Sarah E. Bondos, “Detection and prevention of protein aggregation before, during and after purification”, Analytical Biochemistry 316: 223-231 (2003)
Stephen E. Seinberg, “Erythropoietin Kinetics in Rats: Generation and Clearance”, Blood Vol. 67 (3): 646-649(March 1986).
Sukyung Woo, “Pharmacokinetics and Pharmacodynamics Modeling of Recombinant Human Epo after IV and SC administraion in Rats”, The Journals of Pharmacology and Experimental Therapeutics, Vol. 319(3):1297-1306 (2006).
Sunny Hong Chpel, “A Pharmacodynamics Analysis of EPO Stimulated Reticulocyte Response in Phlebotomized Sheep”, The Journals of Pharmacology and Experimental Therapeutics, Vol 295(1): 346-351 (2000)
Tatsuhiro Ishida, “Accelerated Blood Clearance of PEGylated liposomes following preceding liposome injection: Effects of lipid dose and PEG surface-density and chain length of the first-dose Liposomes”, Journal of Controlled release 105: 305-317 (2005).
“Tetsuro Shingo,” “EPO regulates the In-Vitro and In Vivo production of Neuronal Progenitors by Mammalian Forebrain Neural Stem Cells” “, The Jpurnal of Neuroscience, 21(24):9733-9743 (2001).”
Theresa M. Allen, “Pharmacokinetics of Long-Circulating Liposomes”, Advanced Drug Delivery Reviews 16: 267-284 (1995).
Tianyuan Fan, “Formulation Optimization of Paclitaxel Carried by Pegylated Emulsions Based on Artificial Network”, Pharmaceutical research, Vol. 21 (9): 1692-1697 (2004).
T. M. Allen, “The use of glycolipids and hydrophilic polymers in avoiding rapid uptake of liposomes by the mononuclear phagocyte system”, Advanced drug delivery reviews, 13: 285-309 (1994).
Trubetskoy, V. S. et al., “Polyethyleneglycol based micelles as carriers of therapeutic and diagnostic agents,” S. T. P. Pharma Sciences, 6(1):79-86 (1996).
Trubetskoy, V. S. et al., “Use of polyoxyethylene-lipid conjugates as long-circulating carriers for delivery of therapeutic and diagnostic agents,” Advanced Drug Delivery Reviews, 16:311-320 (1995).
W. Sulowicz, “C.E.R.A (Continuous EPO receptor Activator): A New perspective in anaemia management”, Bantao Journal 4(1): 77-80 (2006)
Weissig, V. et al., “Accumulation of Protein-Loaded Long-Circulating Micelles and Liposomes in Subcutaneous Lewis Lung Carcinoma in Mice,” Pharmaceutical Research, 15(10):1552-1556 (1998).
Woodle, M. C. et al., “Improved Long Circulating (Stealth®) Liposomes Using Synthetic Lipids,” Proceed. Intern. Symp. Control. Rel. Bioact. Mater., 17:77-78 (1990).
Woodle, M. C. et al., “Sterically stabilized liposomes,” Biochimica et Biophysica Acta, 113:171-199 (1992).
Woodle, M. C. et al., “Versatility in Lipid Compositions Showing Prolonged Circulation with Sterically Stabilized Liposomes,” Biochimica et Biophysica Acta, 1105:193-200 (1992).
Yechezkel Barenholz, “Liposome application: Problems and Prospects”, Current Opinion in Colloid & Interface Science 6: 66-77 (2001).
Yoshie Maitani, “Distribution Characteristics of entrapped recombinant human EPO in liposomes and intestinal absorption in rats”, International Journal of Pharmaceutics, 185: 13-22 (1999).
Yoshie Maitani, “Distribution characteristics of entrapped recombinant human EPO in Liposomes and its intsestinal absorption on Rats”, International Journal of Pharmaceutics 185:13-22 (1999).

We claim:

1. A pegylated liposomal unilamellar vesicular composition comprising:
a. a biologically active glycoprotein and/or its pharmaceutically acceptable derivatives; and
b. pegylated unilamellar liposome encapsulating the biologically active glycoprotein and/or its pharmaceutically acceptable derivatives; wherein the liposome is composed of a hydrophilic polymer covalently attached to at least one lipid.

2. The composition as claimed in claim 1, wherein the biologically active glycoprotein and its pharmaceutically acceptable derivatives is selected from erythropoietin and its derivatives.

3. The composition as claimed in claim 1, wherein the hydrophilic polymer is selected from polyethylene glycol and its derivatives, preferably monomethoxy polyethylene glycol.

4. The composition as claimed in claim 3, wherein the monomethoxy polyethylene glycol has a molecular weight of 300-5000 dalton, preferably 1000-2000 daltons.

5. The composition as claimed in claim 1, wherein the lipids are positively charged, negatively charged or neutral.

6. The composition as claimed in claim 1, wherein the lipid is a lipid and/or phospholipid or combination thereof.

7. The composition as claimed in claim 6, wherein the lipid and/or phospholipid is selected from Soy Hydrogenated L-alpha-Phosphatidylcholine (HSPC), Cholestrol, monomethoxy polyethylene glycol conjugated 1,2-Distearoyl-sn-Glycero-3-Phosphoethanolamine (mPEG(n)-DSPE), Ethanolamine phosphoglycerides (EPG) and/or mixture thereof.

8. The composition as claimed in claim 1, wherein the mean distribution size of pegylated liposome is 80-270 nm, preferably it is 90-120 nm.

9. The composition as claimed in claim 1, wherein the composition further comprises neutral surfactants.

10. The composition as claimed in claim 9, wherein the neutral surfactants are selected from polysorbate 80, poloxamer 188 (PLURONIC F68™), Poloxamer 237 (PLURONIC F87), Poloxamer 338 (PLURONIC F108), and/or Poloxamer 407 (PLURONIC F127).

11. The composition as claimed in claim 1, wherein the composition further comprises a buffer system selected from citrate buffer, phosphate buffer, preferably kosmotropes especially ammonium sulfate.

12. The composition as claimed in claim 1, wherein the composition further comprises stabilizers selected from amino acid like L-methionine, L-arginine, Glycine, Histidine, Alanine and salt thereof.

13. A process for preparation of a pegylated liposomal unilamellar vesicular composition comprising the steps of:
i. preparing pegylated liposomal unilamilar vesicles composed of a hydrophilic polymer covalently attached to at least one lipid;
ii. encapsulating a biologically active glycoprotein and/or its derivative within the Pegylated liposome.

14. The process as claimed in claim 13, wherein the encapsulation includes encapsulation, adhering, association of the biologically active protein in/on the surface of the Pegylated liposome.

15. The process as claimed in claim 13, wherein the encapsulation is by means of passive encapsulation or active encapsulation or both.

16. The process as claimed in claim 13, wherein the preparation of Pegylated liposomal unilamilar vesicles is by methods selected from dehydration and rehydration method, by reverse phase, by ethanol injection method or combination thereof.

17. The process as claimed in claim 13, wherein the lipid and the drug are in the ratio of 1:100 to 1:250.

18. The process as claimed in claim 13, wherein the encapsulation of the biologically active glycoprotein is carried out at pH 3.0-8.5, more preferably at pH 3.5 to 7.5.

19. The process as claimed in claim 13, wherein the passive encapsulation is carried out at 2° C. to 40° C., more preferably 4° C. to 37° C.

20. Use of the composition as claimed in claim 1 for the treatment of anemia in human.
